(19) United States
(12) Patent Application Publication
(10) Pub. No.: US 2018/0214575 A1
(43) Pub. Date: Aug. 2, 2018

(54) Composition For Targeted Delivery Of Nucleic Acid-Based Therapeutics

(71) Applicant: Fibralign Corporation, Union City, CA (US)

(72) Inventors: Michael V. Paukshto, Foster City, CA (US); Tatiana S. Zaitseva, San Jose, CA (US)

(21) Appl. No.: 15/747,136

(22) PCT Filed: Jul. 22, 2016
(86) PCT No.: PCT/US16/43762

§ 371 Date: Jan. 23, 2018

Related U.S. Application Data
(60) Provisional application No. 62/196,634, filed on Jul. 24, 2015.

(51) Int. Cl. A61K 48/00; A61L 27/24; A61L 27/54; A61K 38/18; A61K 47/42; A61K 47/10; A61L 27/26; A61L 27/56; A61L 27/58; A61K 9/00

(52) U.S. Cl. CPC A61K 48/0041; A61L 27/24; A61L 27/54; A61K 38/1833; A61K 47/42; A61K 47/10; A61L 2400/06; A61L 27/56; A61L 27/58; A61K 9/0024; A61L 2300/258; A61L 2300/414; A61L 2300/62; A61L 27/26

(57) ABSTRACT

Described herein are gene-activated fibrillar compositions and methods, processes, devices for the design, preparation, manufacture and/or formulation of thereof which are capable to encode at least one polypeptide of interest. Methods treating a disease, disorder and/or condition in a subject by increasing the level of at least one polypeptide of interest, by administering to said subject the gene-activated fibrillar composition are also disclosed.

### RELATED APPLICATIONS

This application claims the benefit of, and priority to, U.S. Provisional Patent Application Ser. No. 62/196,634, filed Jul. 24, 2015, entitled “Composition For Targeted Delivery of Nucleic Acid Based Therapeutics,” the entire disclosure of which is hereby incorporated by reference.

### TECHNICAL FIELD

The present invention relates to gene-activated fibrillar composition and methods of treating clinical conditions using the combination of gene therapy and tissue engineering within a single system. Described herein are gene-activated fibrillar compositions and methods, processes, devices for the design, preparation, manufacture and/or formulation of thereof which are capable to encode at least one polypeptide of interest. The present invention also provides a method of treating a disease, disorder and/or condition in a subject by increasing the level of at least one polypeptide of interest, by administering to said subject the gene-activated fibrillar composition.

### BACKGROUND

The incorporation of nucleic acid vectors (e.g., HGF-mRNA- and HGF-pDNA) into scaffolds with controlled organizations at the nanometer scale has great potential to enhance the interplay between cells and the extracellular milieu since delivery of gene materials to the specific sites introduces signals and cues to cells in a spatial and temporal manner for tissue growth and maintenance. Thus, therapeutic vectors can enhance incorporation of a tissue construct, and its growth and assimilation in the surrounding tissues. Moreover, nanofibrillar matrix used for the delivery of vectors, in particular collagen matrix, can function not only as a targeted carrier and vector complexing agent but also as structural scaffold for tissue engineering application. This combination of gene therapy and tissue engineering within a single system enables a new more comprehensive approach for regenerative medicine. Local gene delivery system using gene-activated matrix integrates these two strategies, serving as an in-vivo local bioreactor with therapeutic gene expression and providing a structural template to fill the lesion defects for cell adhesion, proliferation and synthesis of extracellular matrix.

For almost two decades, major endeavors to develop nucleic acid-based therapeutics have been undertaken. However, clinical applications of this nucleic acid-based therapeutics have been hampered by the low efficiency, off-target effects, toxicity, and inefficient delivery. These limitations have been overcome with the proposed here targeted delivery system and high transfection efficiency of modified mRNA (mmRNA) vectors. Additional factor increasing the transfection efficiency of the proposed composition may be the ability of the delivery matrix to facilitate penetration of vector inside the cell. Thus, the aligned fibrillar substrates enable the solid state transfection.

Nucleic acids are negatively charged molecules such that they do not generally pass through the cell membrane [Akhtar, et. al., Adv. Drug Delivery Rev. 2007, 59, (2-3), 164-182]. The electrostatic repulsion between naked nucleic acids and the anionic cell membrane surface may prevent endocytosis [Akhtar, et. al., Adv. Drug Delivery Rev. 2007, 59, (2-3), 164-182]. Therefore, a selective delivery system is required for efficient transportation of nucleic acids and their release within the targeted cell. The most commonly used gene delivery systems can be divided into biological (viral) and non-biological (non-viral) systems.

Biological carriers and viruses possess and provide efficiency in nucleic acid transfer but are difficult to produce and are toxic [Thomas, et; al., Nat. Rev. Genet. 2003, 4, (5), 346-358]. These limitations mean that development of non-biological systems for nucleic acid delivery remains a high priority. Non-viral delivery systems include peptides, lipids (liposomes), dendrimers and linear or branched polymers with positive charges [Duncan, et al., Adv. Polym. Sci. 2006, 192, (Polymer Therapeutics I), 1-8] that interact with the negatively charged nucleic acids through electrostatic interactions [El-Aneed, J. Controlled Release 2004, 94, (1), 1-14]. Among non-viral delivery systems, dendrimers have the advantage of possessing well-defined structure, size, stability and biocompatibility [Duncan, et al., Adv. Drug Delivery Rev. 2005, 57, (15), 2215-2237]. However, the multistep synthesis and laborious purification at each step of the synthesis, and, consequently, high preparation cost of dendrimers limit their application. Prior art methods to synthesize biomedical polymers rely on a step-growth condensation polymerization protocol that may yield ill-defined polymers with high polydispersity, uncontrolled functionality, topology and composition, which are not ideal for nucleic acid delivery.

Therefore, there is a need for a nucleic acid delivery system that is readily produced and that can be used to efficiently deliver nucleic acids to a targeted biological location to treat various clinical conditions. The solid state transfection using collagenous fibrillar carrier/scaffold is a possible solution where the positively charged collagen fibrils compensate the negatively charged nucleic acid incorporated on the surface of the scaffold.

### SUMMARY OF THE INVENTION

Embodiment of the present invention provides gene-activated fibrillar compositions and methods of treating clinical conditions using the combination of gene therapy and tissue engineering within a single system. Described herein are gene-activated fibrillar compositions and methods, processes, devices for the design, preparation, manufacture and/or formulation of thereof which are capable to encode at least one polypeptide of interest.

Embodiments of the present invention also provides a method of treating a disease, disorder and/or condition in a subject by increasing the level of at least one polypeptide of interest, by administering to said subject the gene-activated fibrillar composition.

### BRIEF DESCRIPTION OF THE DRAWINGS

The accompanying drawings, which are incorporated herein and form part of the specification, illustrate the present invention and, together with the description, further serve to explain the principles of the invention and to enable a person skilled in the relevant art(s) to make and use the invention. Embodiments of the invention are described, by way of example only, with reference to the accompanying drawings, in which:

FIG. 1 is a diagram showing HGF plasmids encapsulated between multiple ultrathin layers of aligned collagen fibrils;

FIG. 2 is a diagram illustrating steps to form a multilayer construct with nucleic acids localized between layers;

FIG. 3 is a diagram illustrating alternative steps to form a multilayer construct with nucleic acids localized between layers;

FIGS. 4A and 4B are schematic representations of the design and application, respectively of scaffold loaded with HGF vector;

FIG. 5 is a schematic illustration of a method of vector loading into a scaffold using an alignment system;

FIGS. 6A-6E are photographs illustrating thread-like collagen scaffolds (also referred to as the “BioBridge”) and characteristics;

FIG. 7A is a cross sectional image of the thread-like collagen scaffold, and FIG. 7B is a graph showing results for two levels of scaffold crosslinking;

FIGS. 8A-8C show various capillarity characteristics of the thread-like collagen scaffold;

FIGS. 9A and 9B illustrate capillarity of the BioBridge at different cross-linked values;

FIG. 10 is a graph showing the force-displacement curve of a BioBridge scaffold in wet state;

FIGS. 11A and 11B are graphs illustrating degradation of the BioBridge by collagenase;

FIGS. 12A and 12B are graphs showing the release of pDNA from the thread-like collagen scaffold with different level of EDC crosslinking and from the lyophilized non-crosslinked thread-like collagen scaffold;

FIGS. 13A and 13B are shows illustrating the transfection efficiency for various embodiments;

FIG. 14 is a schematic diagram illustrating the steps of preparation of a cell migration assay;

FIG. 15 is a schematic diagram illustrating the use of the BioBridge or other scaffolds according to embodiments for prevention of breast cancer related lymphedema after the cancer surgery including lymph node resection and irradiation;

FIGS. 16A and 16B are photographs of human fibroblasts transfected with HGF-mRNA on the surface of aligned collagen scaffold and on the culture tissue plastic;

FIGS. 17A and 17B show a cross section of a micro-carrier and a syringe with injectable micro-carriers.

### DETAILED DESCRIPTION

Recently, the application of mRNA-based technology for pharmacological and regenerative use has been explored. Existing and proposed applications of mRNA-based therapies include cancer immunotherapy, transcript replacement therapy where rate-limiting or defective endogenous proteins are supplemented or replaced, regenerative medicine, and genome editing. In line with the promising application of mRNA in regenerative medicine, successful reprogramming of somatic cells (e.g. fibroblasts) into embryonic-like cells (iPSCs) utilizing mRNA-based techniques has been demonstrated. Furthermore, a more stable, translatable and less immunogenic analog of mRNA, modified mRNA (mmRNA), has been developed and demonstrated ability to be translated into functional proteins indicating the profound clinical potential of mmRNA in delivering therapeutic proteins. The advantages of mRNA over DNA for gene transfer and expression include the high transfection efficiency, the lack of any requirement for nuclear localization or transcription, and the nearly negligible possibility of genomic integration of the delivered sequence.

Scaffold mediated gene delivery provides important advantages for gene transfer including localized delivery of a therapeutic gene, which is mainly taken up by the surrounding cells at the implant site, and gradual vector release from scaffold, which allows for sustained gene delivery, with release rate controlled by the degradation rate of the scaffold material. Another advantage of the scaffold is its role in the protection of the vectors, as it is less likely to be cleared or degraded in vivo when incorporated in a matrix. The scaffold can also act as a bioactive agent for cellular recruitment in situ and a platform for regeneration, providing a template structure on which tissue formation can begin. Implantable polymeric scaffolds define a three-dimensional (3D) space which includes the scaffold and its immediate surroundings, attract cells to migrate and attach to the scaffold, and deliver vectors to the cells located within this space. Cells transfected with scaffold-released vectors at the delivery site secrete protein product acting locally and distributed systemically. In this context the targeted delivery is, at least partially, reversible because the scaffold with impregnated vectors can be removed from the site if it is desired. Multiple vectors can be delivered as well in a desired sequence since the scaffold can be structured as multilayer construct. In addition, the scaffold may have on the surface only ligands for specific cells such that only one type of cells can be bind to the scaffold, e.g., activated T-cells, or specific cancer cells. Thus, only selected type of cells may be subjected to a transfection.

With the main principle in scaffold design being to mimic the natural environment, natural materials used in scaffolds provide lower toxicity in vivo during degradation, lower immune response on implantation, and proper (cell specific) cellular adhesion. Collagen, an essential component of ECM and the main structural protein in the body, is the most widely used natural material in tissue engineering, and is used in wound dressing, sutures, hemostatic agents, skin replacement, bone substitute and blood vessel regeneration. It is preferentially used in its less immunogenic version with removed telopeptide regions, as telopeptides are the main antigenic region in collagen. This type of collagen (atelocollagen) can be used in this application. Collagen-based materials demonstrated a release of plasmid DNA on a scale from hours to several months. For example, systemic effects elicited by DNA incorporated into collagen minipellets administered intramuscularly were significantly longer than direct DNA injection. Collagen-based delivery of nonviral or viral DNA has been employed in models of bone, cartilage, and nerve regeneration; wound healing and muscle repair. All these applications are also targeted here.

Distribution of the vector throughout a 3D space, and transfection on a three-dimensional construct may increase and prolong gene expression level and as compared to 2-D transfection. Furthermore, anisotropy of nanofibrous substrates improves efficiency of reprogramming by lentiviral transduction, which correlates with elongated cell morphology on these substrates [Downing, et al., Nature Materials 2, 1154-1162 (2013)]. Therefore, collagen nanofibrillar scaffold with its anisotropic 3D architecture which induces cell alignment along the fibrils is a good candidate for gene delivery. Many publications demonstrate that nanofibrillar collagen scaffolds support endothelial cell morphology and proliferation in vitro and increase cell survival in vivo. Nanofibrillar scaffold can act as the “depot” of pDNA and mRNA and other bioactive molecules, much like the natural ECM stores and releases growth factors. In addition, scaffolds provide a provisional matrix, e.g., for vessel regeneration. Furthermore, recent data demonstrated that nanofibrillar scaffold reduces fibrosis-related gene expression. The established strategies of collagen zero-length crosslinking by 1-ethyl-3-(3-dimethylaminopropyl)-1-carbodiimide hydrochloride (EDC) can be used for scaffold fabrication. The EDC approach provides the means to control enzymatic degradation of the scaffold by varying the degree of EDC cross-linking without incorporating any additives to the scaffold and without changing the mechanical strength of the scaffold.

Definitions. Here we will use interchangeable “fibril” and “fiber”. We assume that term “nucleic acid” includes the “nucleic acid analogue”. The examples of the devices include: soft tissue repair devices, prosthetic heart valves, pacemakers, pulse generators, cardiac defibrillators, arteriovenous shunts, and stents. Other examples of medical devices, including screws, anchors, plates, staples, tacks, joints and similar devices, for example, are used in orthopedic surgery. These implantable medical devices are made from a wide variety of materials, including, for example, metals, plastics, and various polymeric materials. Other orthopedic devices include implants, such as soft tissue implants, implants for hip, shoulder, elbow and knee replacements and surgeries, or craniomaxillofacial reconstruction, and implant coatings, as well devices used in arthroscopic and laparoscopic procedures. Other examples of medical devices include ocular devices, such as implants, including intraocular lenses and glaucoma shunts. Still other devices include gastrointestinal implants. Further detailed description of various embodiments of the present invention is provided in the following non-limiting examples.

### Example 1

Two EDC/sNHS concentrations for cross-linking (1.0×: 1 mg/ml EDC and 1.1 mg/ml sNHS, and 0.2×: 0.2 mg/ml EDC and 0.22 mg/ml sNHS) can be used for cross-linking of aligned nanofibrillar collagen scaffold. 1.0× and 0.2× crosslinked scaffolds show similar tensile strength, but substantially different degradation rates. The cross-linked scaffolds have been successfully tested for biocompatibility (e.g., BioBridge collagen matrix, 510K device K151083) and for stimulating arteriogenesis in Hind Limb Ischemia model (Nakayama K H, Hong G, Lee J C, Patel J, Edwards B, Zaitseva T S, Paukshto M V, Dai H, Cooke J P, Woo Y J, Huang N F. Aligned-Braided Nanofibrillar Scaffold with Endothelial Cells Enhances Arteriogenesis. ACS Nano. 9(7):6900-8. 2015). Human ECs showed greater outgrowth from aligned scaffolds than from non-patterned scaffolds. Integrin α1 was in part responsible for the enhanced cellular outgrowth on aligned nanofibrillar scaffolds, as the effect was abrogated by integrin α1 inhibition. To test the efficacy of EC-seeded aligned nanofibrillar scaffolds in improving neovascularization in vivo, the ischemic limbs of mice were treated with: EC-seeded aligned nanofibrillar scaffold; EC-seeded non-patterned scaffold; ECs in saline; aligned nanofibrillar scaffold alone; or no treatment. After 14 days, laser Doppler blood spectroscopy demonstrated significant improvement blood perfusion recovery when treated with the scaffold along and with EC-seeded aligned nanofibrillar scaffolds, in comparison to ECs in saline or no treatment. In experiments where ischemic limbs were treated with scaffolds seeded with human ECs derived from induced pluripotent stem cells (iPSC-ECs), systemically injected single-walled carbon nanotube fluorophores were employed to visualize and quantify arterioles using near infrared-II (NIR-II, 1000-1700 nm) imaging after 28 days. NIR-II imaging demonstrated that iPSC-EC-seeded aligned scaffolds group showed significantly higher microvascular density than the saline or cells groups. These data suggest that the “dual” treatment comprised of ECs delivered on aligned nanofibrillar scaffolds improved blood perfusion and arteriogenesis, when compared to cells alone or scaffold alone, and have important implications in the design of therapeutic cell delivery strategies. Thus, the treatment of ischemic limb with the nanofibrillar aligned scaffold demonstrated improvement in blood perfusion and the use of mmRNA-HGF will further enhance this effect.

### Example 2

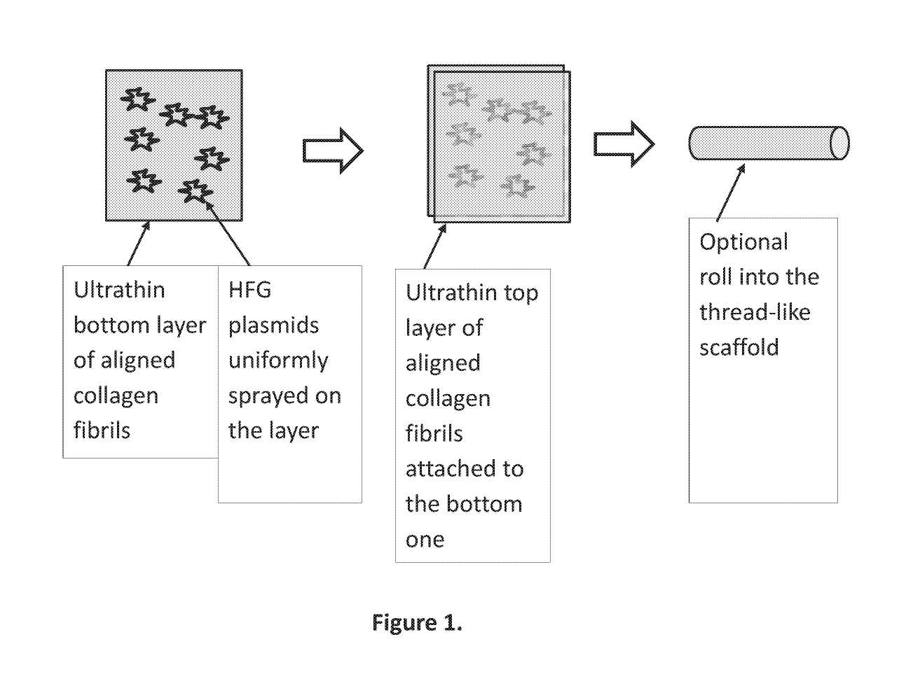

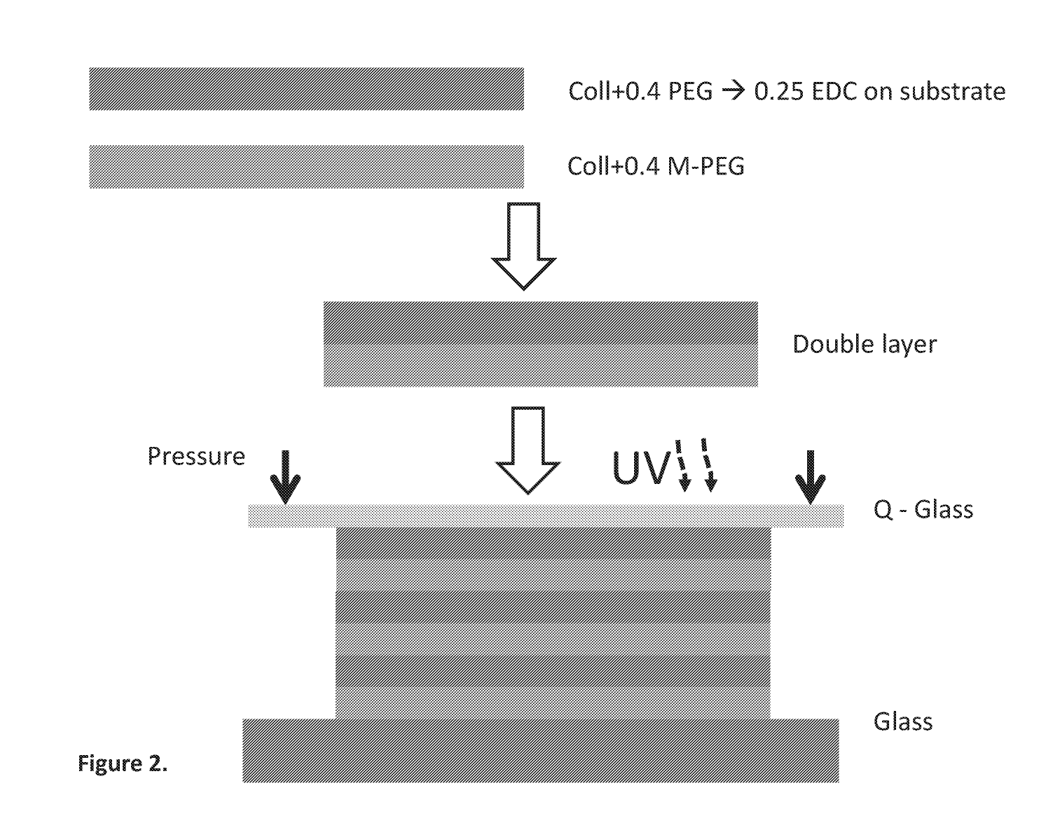

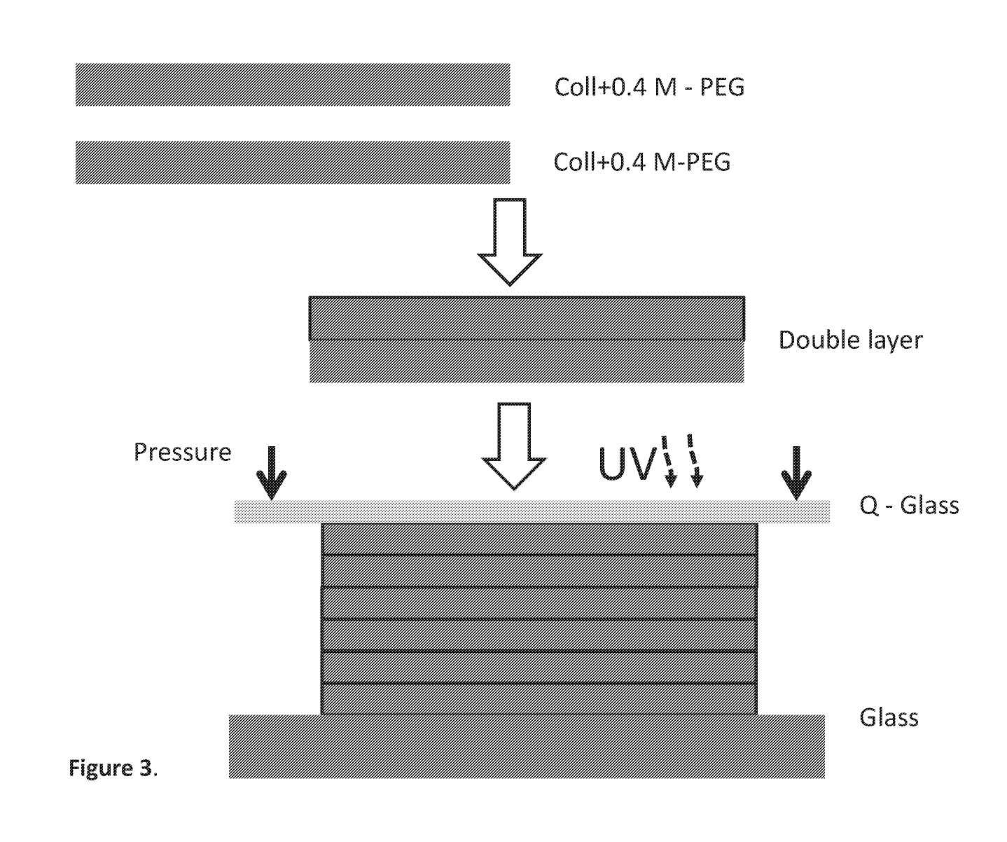

A method for enhancing the transfection efficiency of HGF plasmid DNA in treating and/or preventing angiogenesis-dependent symptoms is proposed. The HGF plasmids will be encapsulated between multiple ultrathin layers of aligned collagen fibrils, see FIG. 1. The thickness and degradation of each layer can be controlled by deposition (layer thickness) and cross-linking, respectively. The typical thickness of aligned collagen layer produced by precise slot-die coater from 50 mg/ml concentrated porcine atelocollagen type I solution can be controlled in the range from 100 nm to 2 microns. The suspension or solution of plasmids (HGF mRNA) will be uniformly sprayed on collagen layer by Sono-Tek precision spray system. Vacuum attachment of multiple layers causes a spontaneous collagen-to-collagen crosslinking and thus encapsulating the plasmids. Thickness of the collagen layer (from nanometer to micron range) and its crosslinking prior the lamination will control the rate of collagen degradation and therefore a release of the plasmids or RNA. Other ways to form a multilayer construct with nucleic acids localized between layers are presented in the FIG. 2 and FIG. 3.

Fibrillar material, e.g., collagen, can be mixed with UV sensitive multi-arm PEG at low concentration then concentrated by evaporation to reach liquid crystal state. We found that:
1. PEG in general and multi-arm PEG in particular does not affect the liquid crystal state. Therefore, all different patterns, in particular, skin-like, aligned, aligned-braided (see U.S. Pat. Nos. 8,492,332B2, 8,227,574B2, 8,513,382B2), can be made from the liquid crystal materials, which include fibrillar collagens;
2. After the material deposition and pattern formation, the film can be cross-linked by UV (e.g., 250 nm, depending on the reactive groups in multi-arm PEG) in dry state.

If the collagen/PEG is deposited on polyethylene terephthalate (PET) substrate, then this layer will be cross-linked to the PET substrate.

If UV-mask is used during the UV crosslinking, then uncross-linked water soluble collagen can be removed by water rinsing (with the pH in the range 2-6).

The deposition and cross-linking steps can be repeated in order to form patterned multi-layer stack. Different nucleic acid formulation can be deposited between the layers before crosslinking.

The collagen/PEG can be coated on the substrate which is not crosslinked by UV, e.g., glass substrate. Then each layer can be peeled-off after crosslinking. Alternatively, the collagen/PEG layers can be peeled-off before cross-linking in order to form multilayer stack, see FIG. 2 and FIG. 3. Here, Q-glass is a quartz glass plate transparent for 250 nm UV radiation. “Col.+0.4 PEG” means molecular collagen mixed with 0.4% PEG by weight. “0.25 EDC on substrate” means a specific EDC crosslinking while the deposited film is attached to a substrate (e.g., PET substrate). After the EDC cross-linking the collagen can be peeled-off from the substrate (EDC does not cause a crosslinking between collagen and PET). “M-PEG” means multi-arm PEG that can be cross-linked by UV in dry state.

The additional material which does not change liquid crystal state of molecular atelocollagen in acidic pH is EDC/NHS. After the deposition of the collagen/EDC/NHS to form nanoweave structure the film can be cross-linked by changing the pH (e.g., in ammonia vapor).

The aligned nanofibrillar collagen scaffold will induce the elongation and migration of a cell which will populate the scaffold after its implantation. In this way we are able to mimic a native physiological environment. In addition, we deliver a suitable nucleic acid to the cells to enhance a desired outcome, e.g., regeneration. This is the method of a local sustained cell expression over a prolonged period of time determined by the collagen crosslinking (from 4-6 weeks to several months).

### Example 3

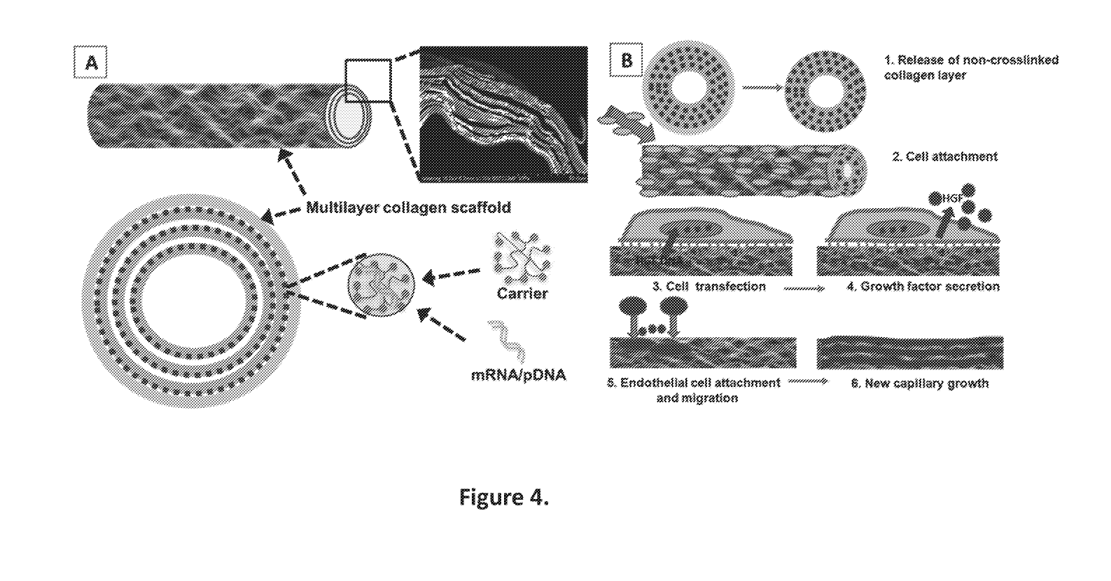

Schematics of the design FIG. 4A and application FIG. 4B of scaffold loaded with HGF vector. In the presence of the aligned collagen, fibroblasts, local endothelial and endothelial precursor cells residing or attracted to ischemic region could attach to the scaffold, produce HGF, and stimulate formation of capillaries.

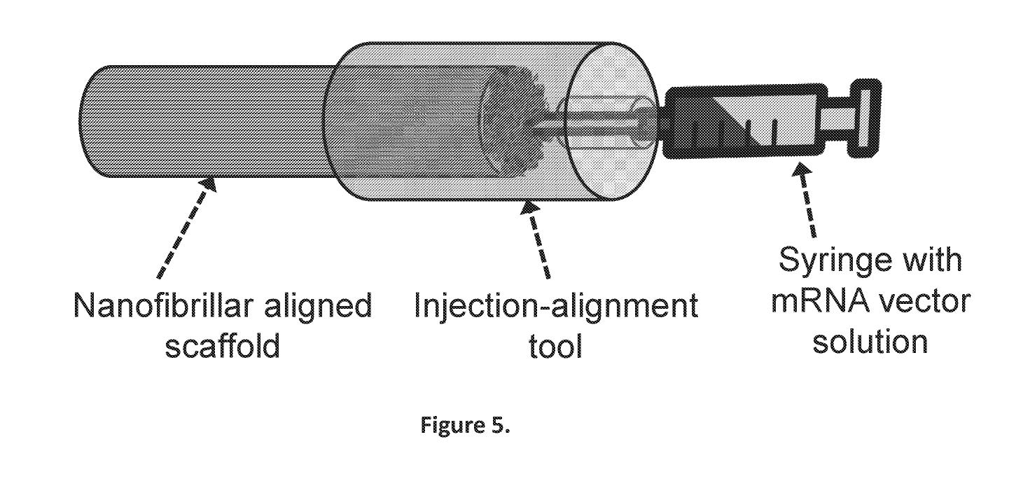

The method of vector loading into a scaffold uses the alignment system schematically shown in the FIG. 5. The thread-like porous scaffold is inserted into a transparent tube and hold there by a clamp. A second smaller transparent tube is aligned with the first one and syringe is inserted into the second tube. In this way the vector solution can be precisely delivered into the scaffold using its porosity and capillarity. The addition of a dye to the vector solution (e.g., methylene blue) simplifies the loading method. Lyophilization further stabilizes the adhesion of the nucleic acid to the scaffold surface. Addition of the mono- or polysaccharides into the vector solution protect the nucleic acid during the lyophilization at low temperature (less than −20 C) and high vacuum (less than 50 mPa). The proposed method is suitable for the use in both aseptic conditions and the surgery room environment.

### Example 4

We have fabricated BioBridge scaffold made of fibrillar collagen according to methods disclosed in the U.S. Pat. Nos. 8,492,332B2, 8,227,574B2, 8,513,382B2.

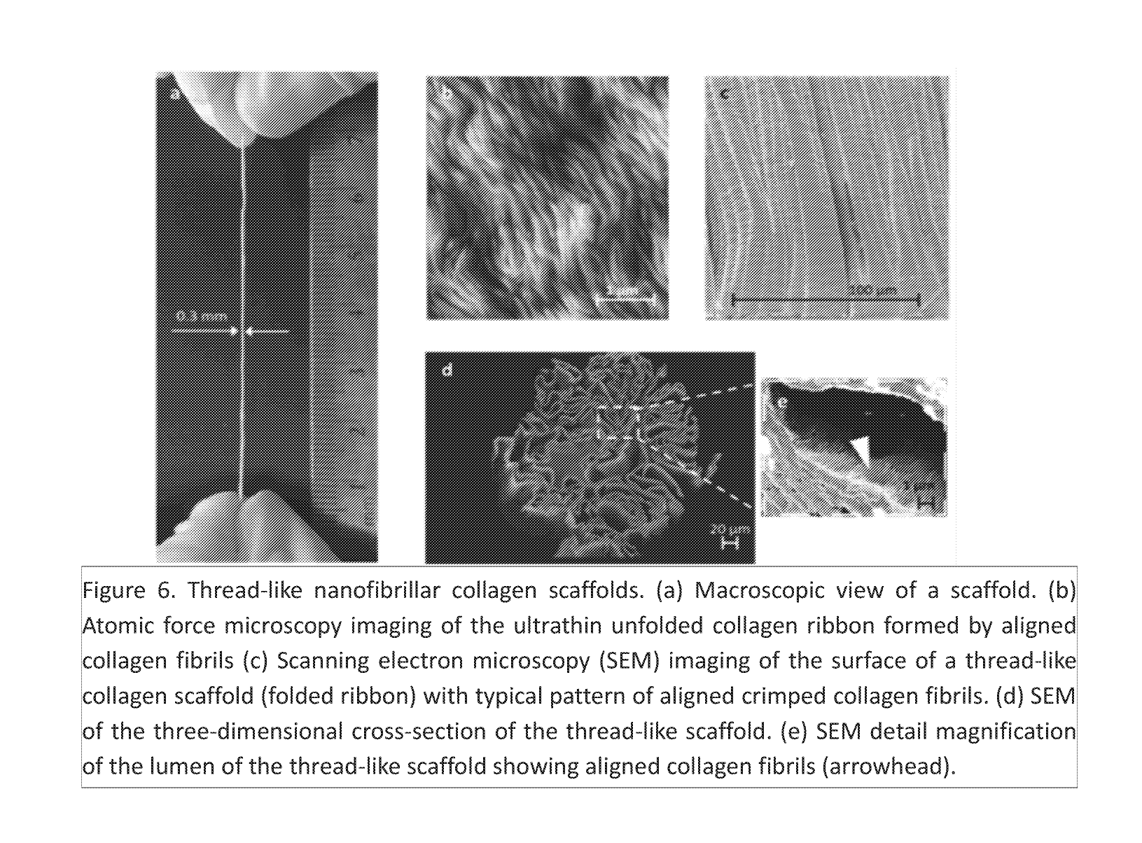

Characterization of BioBridge. The basic characteristics of the thread-like collagen scaffold (BioBridge) used in this example are shown in FIG. 6. BioBridge is formed from a folded ultrathin collagen ribbon such that all thin collagen fibrils forming the ribbon are aligned along the scaffold direction.

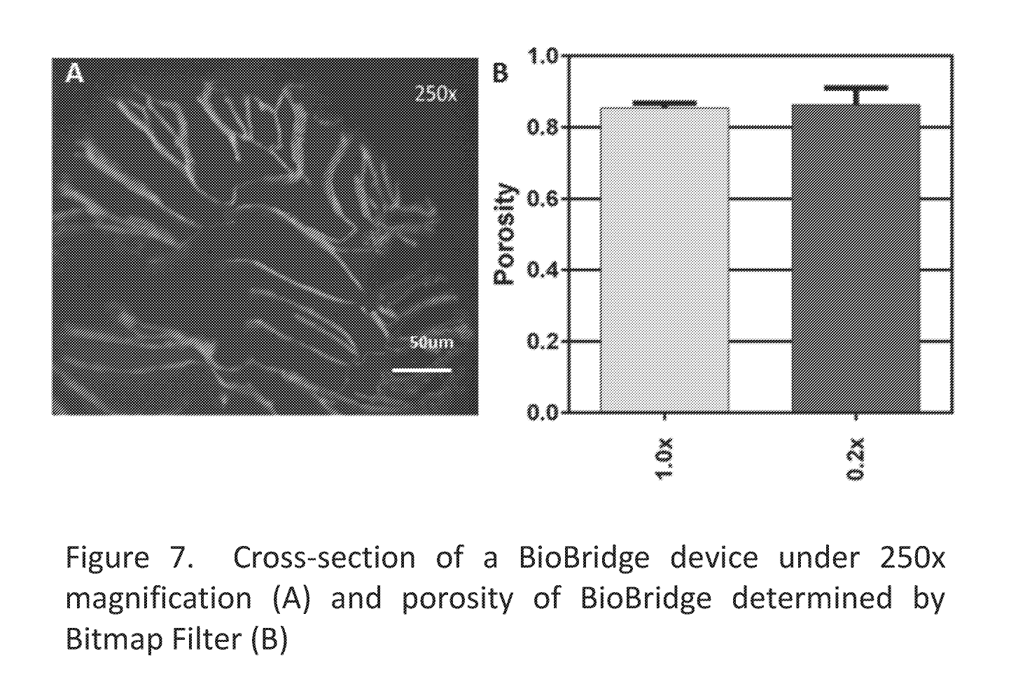

BioBridge scaffold porosity. The cross-section image of BioBridge scaffold was taken by high resolution reflective microscope. The typical image is presented in FIG. 7A. The image was analyzed by a standard bitmap filter for the porosity value by taking a ratio between black pixels and total cross-section area. The results are presented in FIG. 7B for two levels of the scaffold crosslinking: 1.0× and 0.2×. The average porosity is about 85% with low standard deviation.

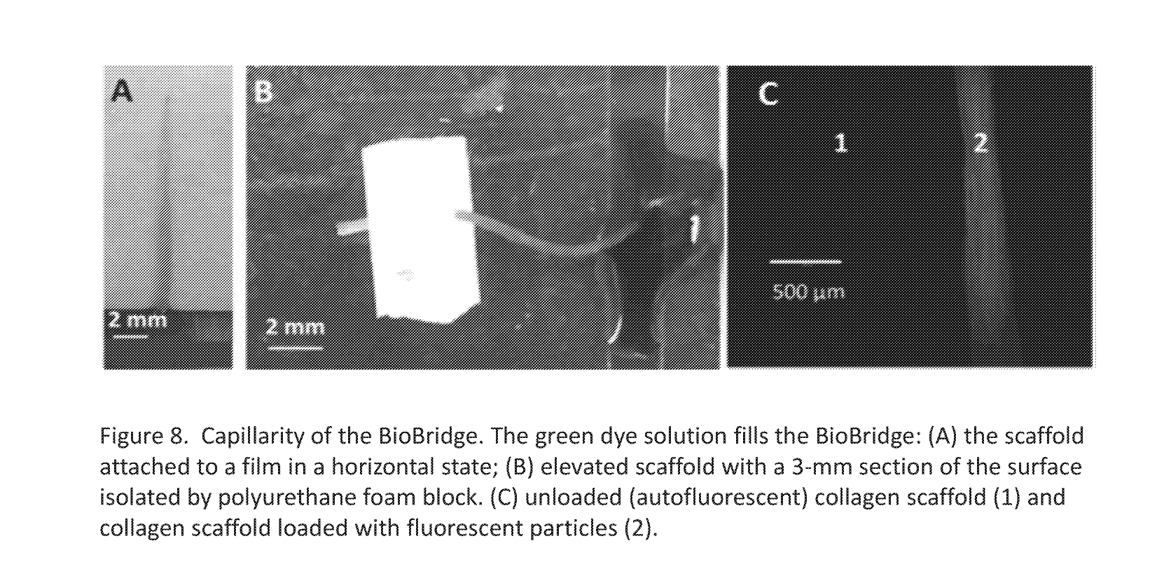

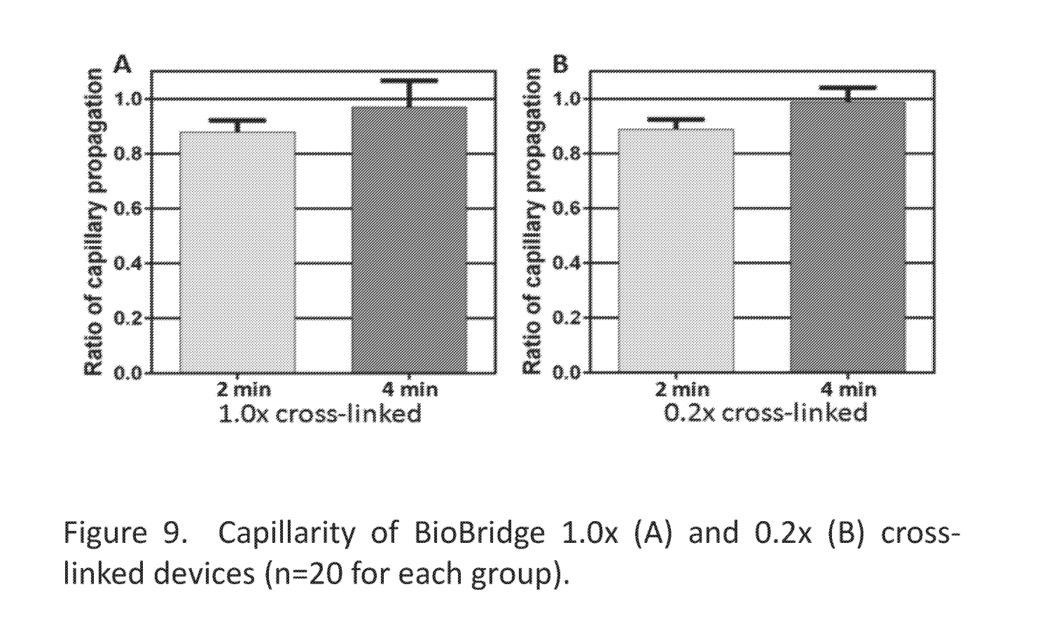

BioBridge scaffold capillarity. The capillarity of the scaffold was measured for 13-mm long sections of BioBridge attached to a double scotch tape in a horizontal position, see FIG. 8A and FIG. 8B. A syringe and a 25 G needle were used to load approximately 0.1 mL of green food coloring dye on each BioBridge end. Time points of 2 and 4 minutes were taken to analyze how far the dye had traveled up for each section. The capillary propagation of the green dye was measured in green channel with the baseline of the initial white color of the dry collagen. The ratio of the dye propagation distance to the total distance of the BioBridge section is presented in the FIG. 9 for each experiment. The results demonstrated high capillarity of the BioBridge with low standard deviation. The 0.2× cross-linked BioBridge has slightly higher capillarity than 1.0× cross-linked BioBridge which is consistent with the porosity measurements. It takes about 4 minutes for pressureless dye propagation through the 13 mm section of BioBridge.

The BioBridge device has high porosity (about 85%) suitable for drug loading. The pores are interconnected to allow capillary flow along the device. The porous structure of BioBridge (FIG. 6) provides for capillary properties which can be used to load HGF plasmids (HGF-pDNA) into the scaffold.

In order to optimize the pDNA loading, we used as a model the fluorescent spheres of 100 nm and 1 μm in diameter. The actual plasmid size is somewhere in between these two diameters. Solutions of fluorescent nano- and microspheres were prepared at the concentration of 0.1 mg/ml and the spheres were loaded into the scaffold by capillary flow. The loading time was the same for both types of particles. The presence of particles was observed by fluorescent microscope Leitz DM IRB (FIG. 8C).

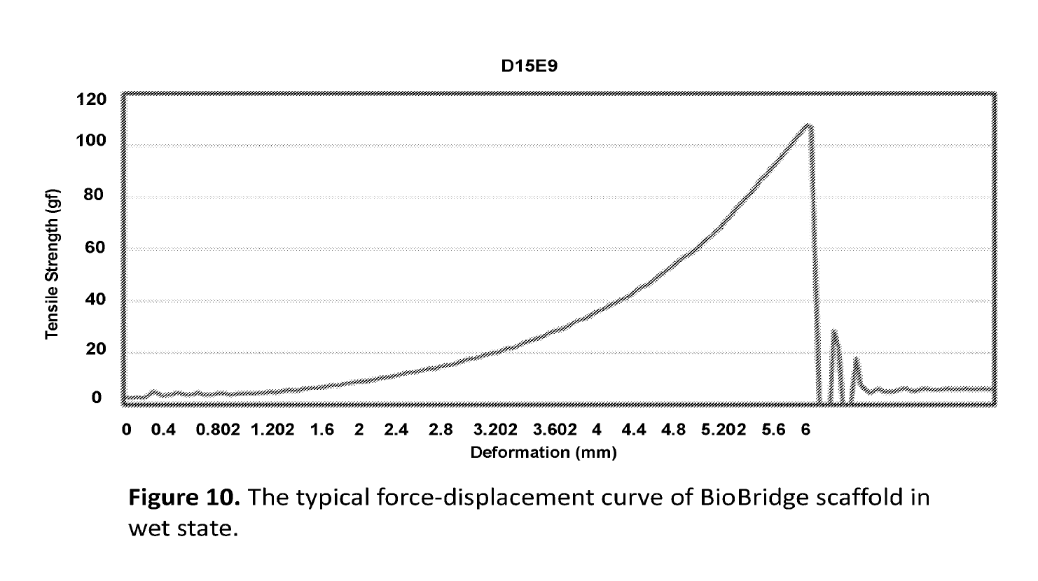

Mechanical properties of BioBridge. The BioBridge scaffold is made from a 1 micron (1×10−6 m) thick membrane which is collapsed length-wise to form a thread-like structure. The cross-sectional area of all BioBridge scaffold is equal to 2.54×10−8 m2. The stress is calculated according to the formula: Stress (MPa)=10−6× Force (N)/Area (m2). Mechanical measurements of the BioBridge were conducted by calibrated tensile tester in the wet state. The typical force-displacement curve is presented in the FIG. 10. Thus, the typical tensile strength is higher than 100 gF and therefore the maximum stress is more than 30 MPa.

Enzymatic Degradation of BioBridge. Collagen scaffold degradation in collagenase has been assayed by measuring soluble protein using ninhydrin reactivity [Starcher B. A ninhydrin-based assay to quantitate the total protein content of tissue samples. AnalBiochem 292: pp. 125-129. (2001)]. For each sample, 1-cm scaffold segments were placed in a microcentrifuge tube. The samples were incubated at 370 C in 200 μL of 0.1 M Tris-base, 0.25 M CaCl2 (pH 7.4) containing 100 or 50 U/mL bacterial collagenase (Clostridium histolyticum, Calbiochem). After incubation, the samples were centrifuged at 15,000 RCF for 10 min and the supernatant was reacted with 2% ninhydrin reagent (Sigma) in boiling water for 10 min. The optical density (OD) was then measured at 570 nm in a spectrophotometer (SpectraMax, Molecular Devices, Sunnyvale, Calif., and the relative OD was calculated by subtracting the value of the background (collagenase only control) from the acquired optical density. Final data were represented as relative OD per mg of material.

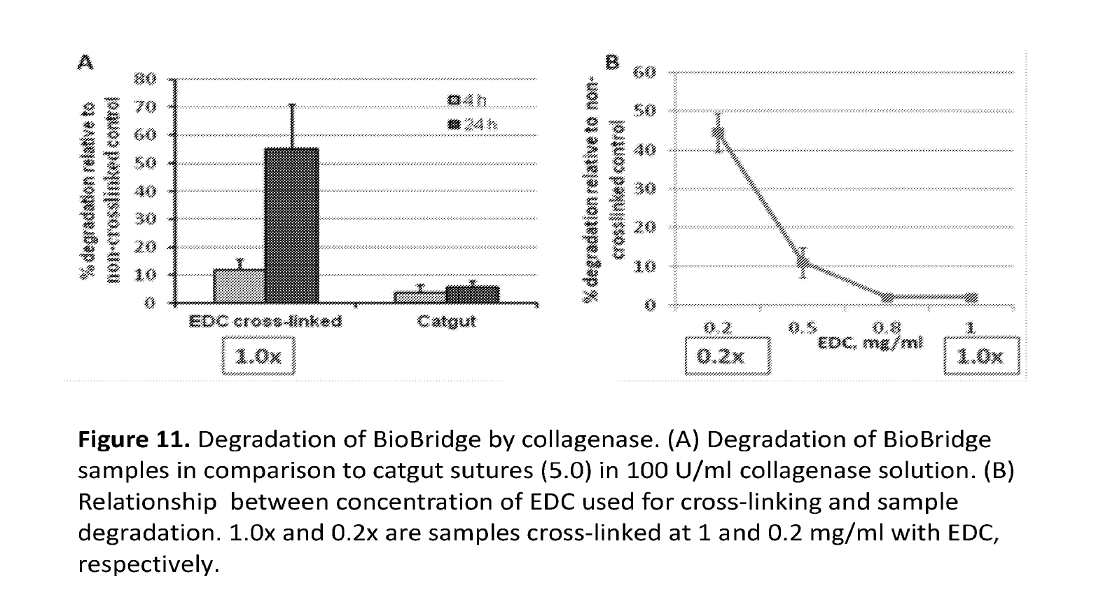

Our data showed that Biobridge 1.0× was degraded by collagenase faster than catgut suture (FIG. 11A), which specifications indicate that it is degraded by 3 months post-implantation. Our data have also demonstrated that BioBridge degradation is a function of EDC concentration used for cross-linking (FIG. 11B), and Biobridge 0.2× was degraded by collagenase faster than 1.0×.

Preparation of pDNA-supplemented scaffold. To evaluate the amount of DNA to be loaded into scaffold by capillary force, we first used the 0.2× porous scaffolds described above to incorporate the plasmid by incubating the scaffold in the solution of pCMV6-AC-GFP plasmid. Scaffold samples (5-mm long) were incubated in a 40 μl aliquot of plasmid solution (at 1 to 0.025 μg/μl concentration, 4 to 40 μg total DNA amount in the reaction) at room temperature for 1 h, then the pDNA solution was removed and replaced with 500 μl of cross-linking solution (EDC, 1 mg/ml and sNHS, 1.1 mg/ml in PBS at pH 6.0) for 30 min. The pDNA-scaffold was rinsed 4 times in PBS for 30 min.

Increasing pDNA amount in the reaction from 4 to 40 μg did not significantly increase the amount of DNA retained in the scaffold. Our pilot data showed that a substantial amount of DNA (up to 208 ng) is retained in the scaffold after its incubation with 4 μg DNA, which constitutes 5% of the DNA amount introduced into the reaction. While the retained DNA amounts did increase with increasing the DNA content in the reaction mix, the amount of DNA retained in the scaffold did not increase proportionally. Of the 40 DNA added to the reaction, 295 ng (0.7%) was retained. Our scaffold was able to retain up to 566 ng per mm3 scaffold volume, which is higher than the amount of pDNA retained by a collagen scaffold when normalized to mm3 scaffold volume based on the data reported in the literature (70 ng) for a similar approach. Therefore, we proceeded with using 4 total pDNA amount in the reaction.

The pDNA-retaining and releasing capabilities of the scaffold were further explored with modifications of post-loading procedure, as we used (1) decreased concentration of EDC/sNHS in the reaction, and (2) lyophilization of the pDNA-loaded scaffold instead of EDC-cross-linking. All scaffold samples (5-mm long) were incubated in a 40-μl aliquot of plasmid solution (at 0.025 μg/μl concentration) at room temperature for 1 h, then the DNA solution was removed. It was replaced with 500 μl of cross-linking solution (EDC, 1 mg/ml and sNHS, 1.1 mg/ml (1.0×) or EDC, 0.2 mg/ml and sNHS, 0.22 mg/ml (0.2×) in PBS at pH 6.0) for 30 min. The pDNA-scaffold was rinsed 4 times in PBS for 30 min. Alternatively, after pDNA loading scaffold samples were frozen and lyophilized (Lyo).

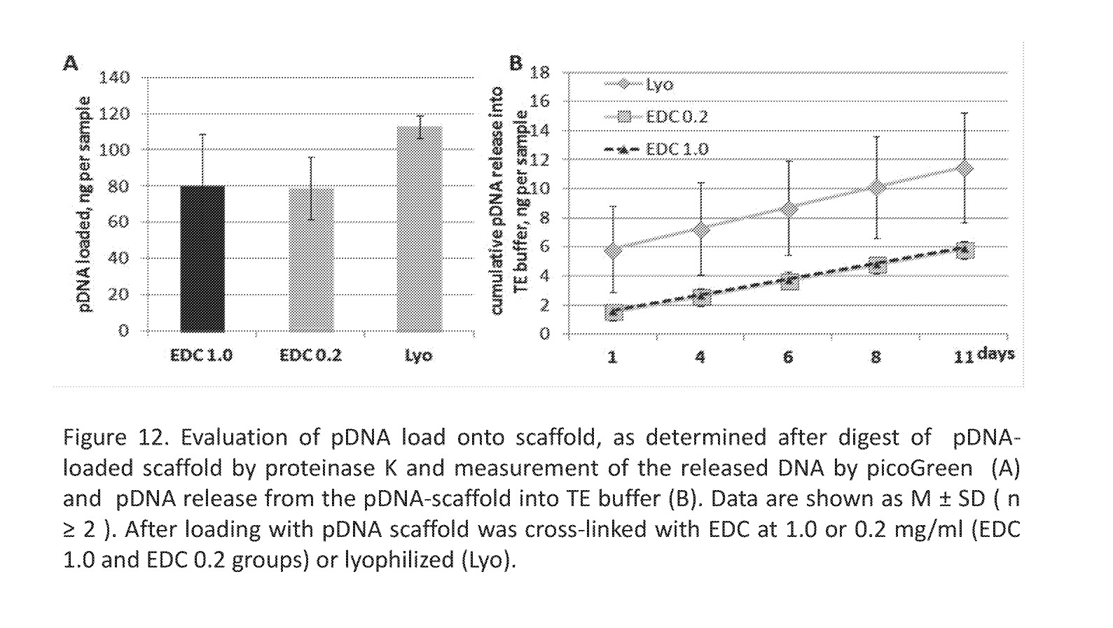

Evaluation of pDNA incorporation into the scaffold. To evaluate the amount of DNA attached to the scaffold, pDNA-scaffolds prepared as described above were subjected to digestion in 50-μl aliquots proteinase K solution (Roche) at 54° C. for 18 h. DNA content in the digest samples was aliquots was assessed using PicoGreen reagent (Quant-iT™ PicoGreen® dsDNA Assay Kit, Life Technologies, NY) following manufacturer's instructions. The fluorescence intensity was measured by fluorescence plate reader Analyst HD&AT (LJL Biosystems Inc.). We have found that the reduction in EDC concentration did not substantially change the amount of pDNA retained in the scaffold, however lyophilization increased the pDNA retention (FIG. 12A).

Analysis of pDNA release. To assess the release of pDNA from the scaffold, pDNA-scaffolds were incubated in 40-ul TE buffer aliquots, and the aliquots were collected at specific intervals and replaced with fresh aliquots. DNA content in collected samples was assessed using PicoGreen reagent as described above. As shown in FIG. 12B, pDNA-scaffolds stably release small amounts DNA into TE buffer through day 11. The total amount of DNA released through day 11 of incubation was 5.9 ng, or 9.7% of the pDNA incorporated (61 ng) for “EDC 1.0” scaffold, 5.8 ng, or 8.3% of the pDNA incorporated (70 ng) for “EDC 0.2” scaffold, and 11.5 ng, or 10.3% of the pDNA incorporated into the scaffold (112 ng) for “Lyo” scaffold. Based on these data, we concluded that lyophilization may be used to load scaffolds for transfection experiment.

Optimization of plasmid transfection efficiency. To determine the optimal composition of the pDNA loading mixture, we first identified the optimal transfection agent for pCMV6-AC-GFP plasmid. We used (1) TurboFectin 8.0 (OriGene) following the plasmid manufacturer's standard protocol, and (2) alternative transfection agents (Viromer) recommended by the plasmid manufacturer. Of the two versions of Viromer (Red and Yellow) initially tested, Viromer Red resulted in higher transfection efficiency and was used for further optimization using both standard and direct complexation transfection protocols suggested by the manufacturer (LipoCalyx). Human foreskin fibroblasts were grown in DMEM supplemented with 10% FBS on 96-well tissue culture plates to reach 60-80% confluence. Plasmid DNA was diluted into serum-free medium without antibiotics, gently mixed, combined with transfection agent, incubated for 15-45 minutes at room temperature, and added to the cell culture. Cells were maintained at 370 C in a 5% CO2 incubator before testing for effects of overexpression starting from 24-48 h. GFP gene expression was monitored periodically by fluorescence microscopy (Leica DMIRB). Transfection using TurboFectin, even after several iterations of optimization steps, only resulted in a low fraction of GFP-expressing cells. Transfection with standard protocol for Viromer Red gave higher number of GFP-expressing cells, and transfection with direct complexation protocol resulted in the highest number of GFP-expressing cells.

GFP mRNA.

We considered that mRNA may be an efficient alternative to pDNA vector in the final product, as it provides for efficient transfection, therefore explored the feasibility of using GFP mRNA (RNAcore, Houston Methodist Research Institute, TX) for transfection in our studies, and proceeded with evaluation of its transfection efficiency in comparison with pDNA.

Evaluation of pDNA and mRNA transfection efficiencies. (1) pDNA transfection: We continued transfection studies with pCMV6-AC-GFP using direct complexation protocol with transfection reagent Viromer Red (described above), which showed the highest transfection efficiency in our optimization experiments. Human foreskin fibroblasts were grown in DMEM supplemented with 10% FBS on 96-well tissue culture plates to reach 60-80% confluence. Plasmid DNA was diluted into serum-free medium without antibiotics, gently mixed, combined with transfection agent, and incubated for 15-45 minutes at room temperature. (2) mRNA transfection: We used the transfection reagent recommended by manufacturer, Lipofectamine RNAiMAX (Invitrogen, Cat#13778-075), and Viromed Red (following direct complexation protocol similar to the one described for pDNA transfection).

Lipofectamine transfection protocol. We followed the basic steps of the protocol suggested by the manufacturer, with adjustment from 6-well plate format to 96-well format. Lipofectamine (1.6 or 2.4 μl) was diluted in OMEM (12 or 17.6 μl), and added to mRNA (1.3 or 2 μl of 50 ng/μl), diluted in OMEM (12 or 18.4 μl). The Lipofectamine/mRNA mix was incubated at room temperature for 15 min (at this time, growth media were replaced with OMEM), then added dropwise to each well, 3.4 or 5 ul per well, which resulted in 8.3 or 12.5 ng mRNA introduced per well. After 4 h of incubation OMEM was removed and replaced with DMEM/10% FBS.

Viromer Red Direct Complexation Protocol.

Working mRNA solution (50 ul) was prepared at 15 ng/ul. Stock Viromer solution (0.3 ul) was placed in a tube, and mRNA solution was added directly to the tube with Viromer, gently mixed, and incubated at room temperature for 15 min. At this time point, growth media were refreshed. Transfection mix was applied to the cells added dropwise to each well, 6.7 ul per well, which resulted in 100 ng mRNA introduced per well. Further optimization included reducing the mRNA amount in the reaction by using 5 ng/ul working solution or adding 2 ul of the transfection mix prepared with 15 ng/ul working solution. Cells were maintained at 37° C. in a 5% CO2 incubator, and GFP gene expression was monitored periodically by fluorescence microscopy (Leica DMIRB).

Transfection efficiency evaluation. At 24 h post-transfection, GFP expression was assessed by fluorescence microscopy of live culture. After representative images were taken, cells were fixed with 2% paraformaldehyde and stained with Hoechst (1:10000) for nuclei quantification. For each well, 3-4 matching images were taken for GFP and nuclei. Number of nuclei per image was calculated using Amscope software, and number of GFP-positive cells was counted manually using the grid superposed onto the image. Transfection efficiency was calculated as (number of GFP-positive cells/number of nuclei)×100. The level of GFP expression was further assessed by measuring GFP fluorescence intensity in the fluorescence plate reader.

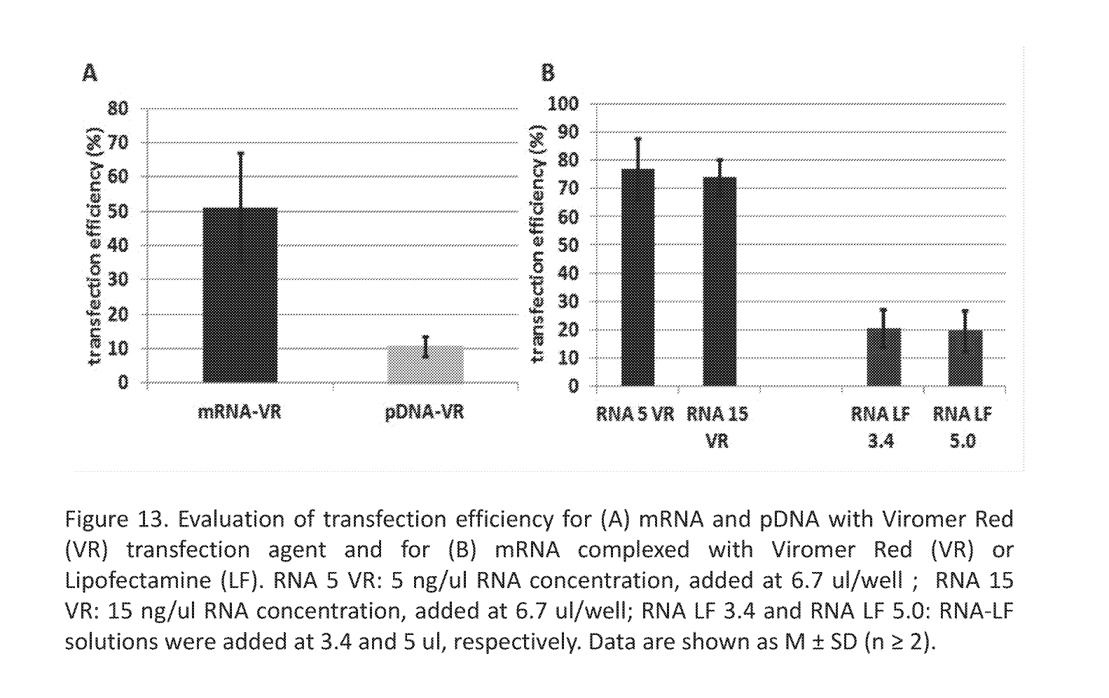

Comparison of transfection efficiencies between mRNA and pDNA has shown that for the conditions used, mRNA was more efficient in transfecting fibroblasts (FIG. 13A). Further, we extended the range of conditions for mRNA transfection (FIG. 13B), and demonstrated that Viromer Red transfection agent provided a higher transfection efficiency, for all variations used, than Lipofectamine. It should be noted that as we based our initial protocols on manufacturer's recommendations, the amount of mRNA introduced into the well varied between Viromer and Lipofectamine protocols.

3D Cell Migration Assay.

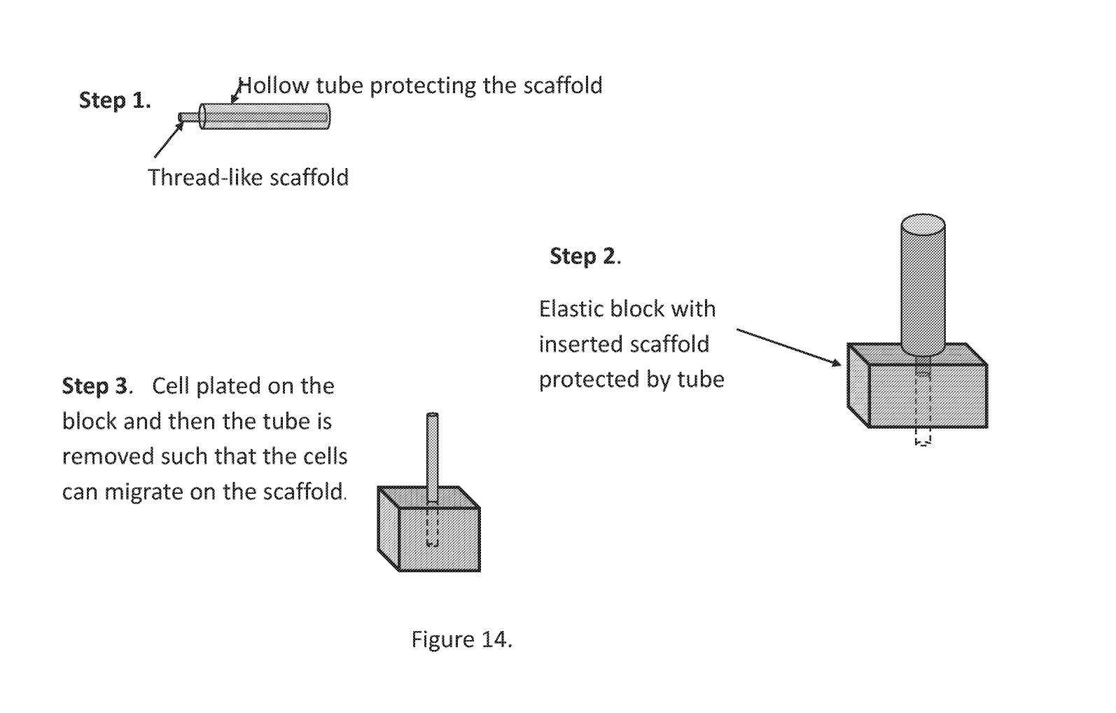

The cell migration assay has been developed to test an effect of growth factor release to cell migration. The schematic diagram of this assay is presented in the FIG. 14.

Applications.

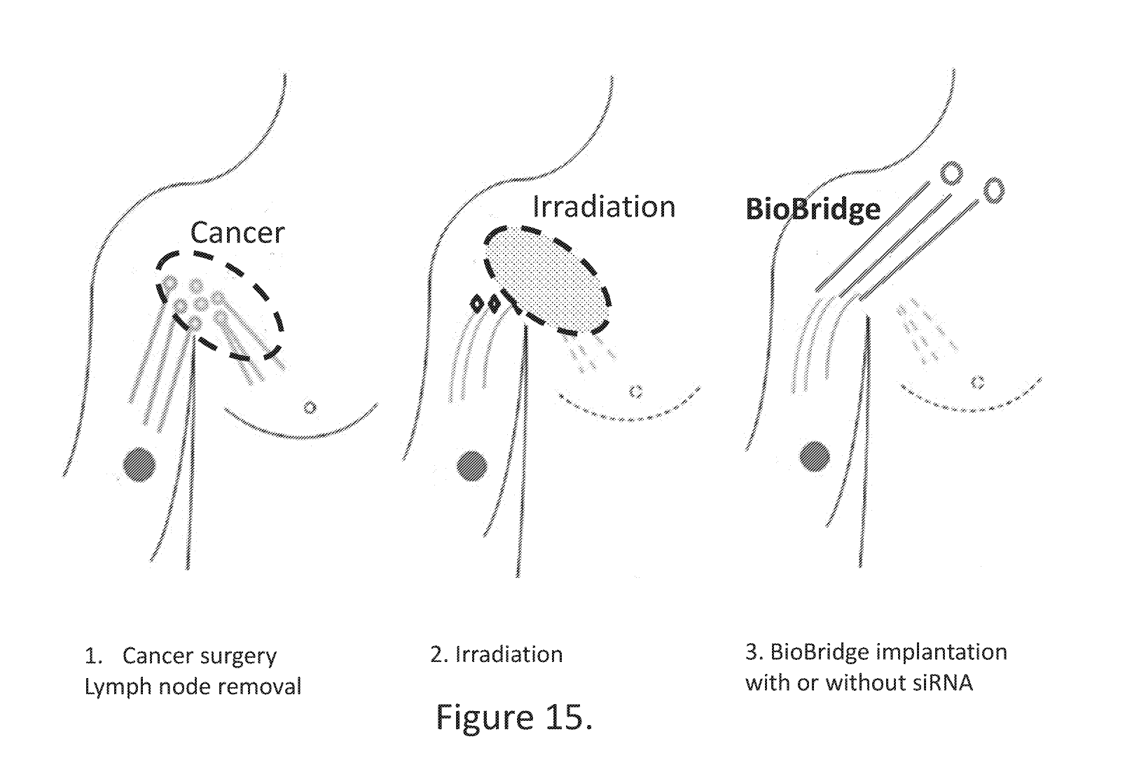

The above methods can be used to make the means for treatment lymphedema, glaucoma, keloid and other scars, cornea corrections, dental disorders—by nanopatterned gene activated scaffolds for targeted gene delivery to modulate local cell response including cell differentiation. The use of the BioBridge or other scaffold which can direct formation of lymphatic vessels to reconnect the disrupted lymphatic system is presented in FIG. 15. It can be supplemented by gene materials preventing the cancer cell development, e.g., siRNA. This method may prevent lymphedema formation and may be used during or immediately after the cancer surgery.

### Example 5

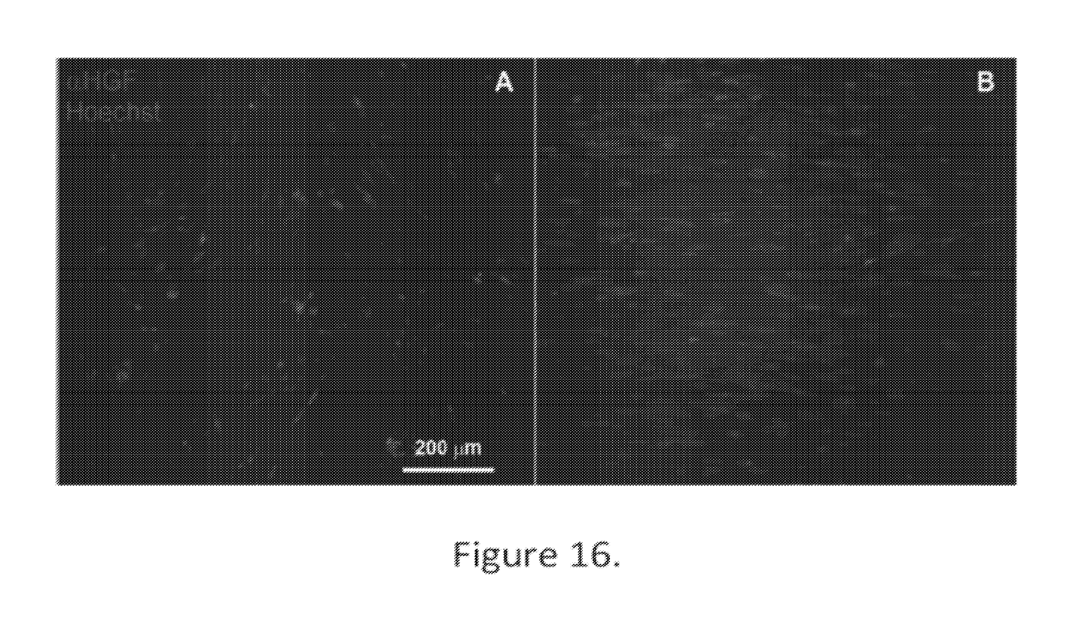

Human fibroblasts transfected with HGF mRNA and stained with α-hHGF (red) and Hoechst (blue) are presented in the FIG. 16, where A—cells were seeded on tissue culture plastic; B—cells were seeded on aligned collagen scaffold (aligned-crimp coating on plastic). In vitro transfection of human T-cells on a TERT mRNA-loaded scaffold may significantly increase their proliferative potential, and therefore, the overall efficacy of T-cell therapy.

### Example 6

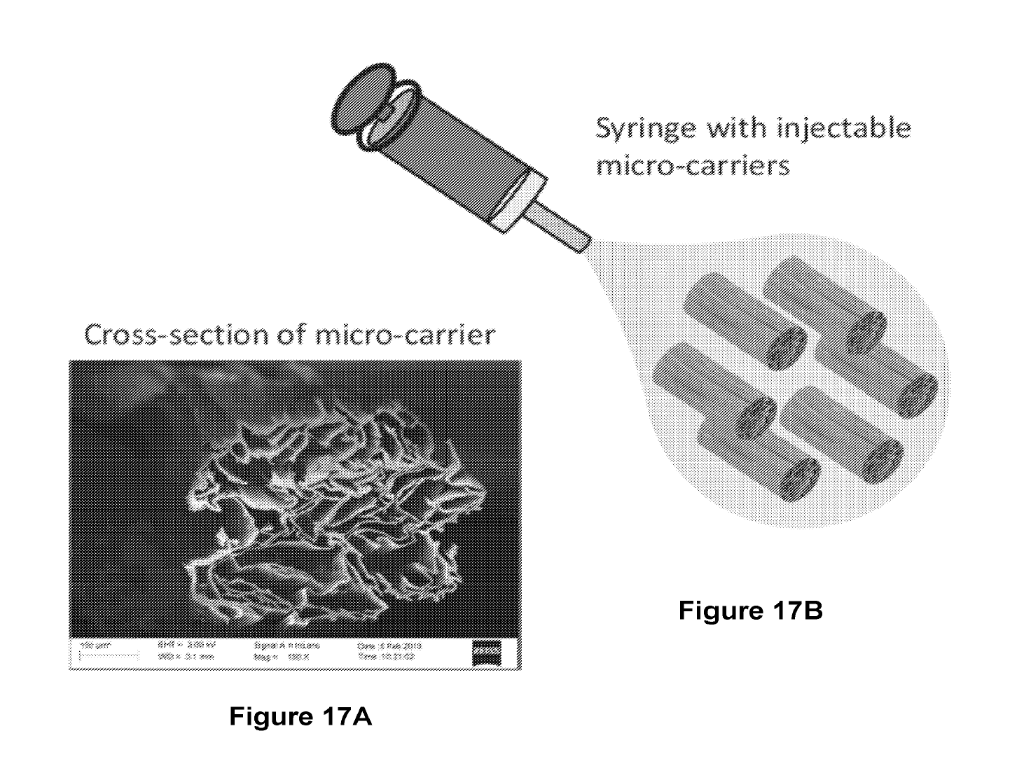

Micro-carrier platform for cell/mRNA delivery. Injectable nanoweave micro-particles with diameter from 50 to 300 microns can be made by cutting from single BioBridge scaffold. For example, such micro-particles can be made from aligned thread-like collagen scaffold described in the Example 4. In this case the micro-particles will have aligned collagen structure (see FIG. 17) with large surface area, multilumenal porosity, and tuned degradation. These micro-particles can be activated by nucleic acid using capillarity or being introduced in the initial collagen solution before BioBridge formation. Such micro-carriers are injectable by at least positive displacement syringe and enable sustain delivery of nucleic acids (e.g., mRNA or pDNA). In particular, these micro-carriers may be injected into lymph nodes and target specific cancer cells by releasing nucleic acid vectors encoding for factors instrumental in preventing cancer cell from spreading and proliferating.

Exemplary embodiments have been described with reference to specific configurations. The foregoing description of specific embodiments and examples has been presented for the purpose of illustration and description only, and although the invention has been illustrated by certain of the preceding examples, it is not to be construed as being limited thereby.

What is claimed is:

1. A composition comprising at least one aligned fibrillar material enabling attachment and alignment of at least one type of cells; and said fibrillar material comprises nucleic acid based molecules that modulate gene expression of the cells attached to the fibrillar material.

2. A composition of claim 1, wherein the nucleic acid based molecules are the nucleic acid vectors that enable transfection of the cells attached to the fibrillar material.

3. A composition of claim 1, wherein the fibrillar material is selected from the group of self-assembled polypeptides forming liquid crystal material, fibrillar polypeptides, fibrillar collagens, fibrin, fibronectin, laminin, silk, poly-L-lactic acid, polyglycolic acid, elastin-like polypeptides, poly(propylene carbonate), chitosan, their derivatives, or any combination thereof.

4. A composition of claim 1, wherein the fibrillar material is biocompatible biodegradable material with tunable rate of degradation.

5. A composition of claim 1, wherein the composition further comprises a medical device.

6. A composition of claim 1, wherein the nucleic acids is selected from the group of pDNA, mRNA, miRNA, mmRNA, siRNA, RNAi, ssRNA, pDNA, dsRNA, antisense, catalytic RNA, and their derivatives.

7. A composition of claim 1, wherein nucleic acid is at least partially encapsulated by the cationic region of the polymeric nanostructure, or cationic lipids and/or cationic polymers that form electrostatic complexes with the nucleic acids.

8. A composition of claim 2, wherein chemical transfection methods rely on electrostatic interactions to bind nucleic acid and to target cell membranes utilizing compounds such as calcium phosphate, polycations, liposomes, as well as cationic lipids, polymers, dendrimers, nanoparticles, polyethylenimine, and polylysine.

9. A composition of claim 1, wherein the nucleic acids or nucleic acid based compounds modulate gene expression of the cells selected from the group consisting of hepatocytes, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, pancreatic beta cells, pituitary cells, synovial lining cells, ovarian cells, testicular cells, fibroblasts, B cells, T cells, reticulocytes, leukocytes, granulocytes and tumor cells.

10. A composition of claim 1, wherein nucleic acid based molecules is loaded into the fibrillar material by injection using alignment system.

11. A composition of claim 1, wherein the fibrillar material further comprises poly(ethylene glycol), carbohydrates, glycoprotein, glycosaminoglycan, monosaccharides, polysaccharides, or their combinations.

12. A composition of claim 2, wherein the transfection of the cells attached to the aligned fibrillar material is at least 60% higher than the transfection of the same cells attached to a tissue culture plastic.

13. A composition forming a thread-like fibrillar scaffold with the fibrils aligned in the direction of the thread such that the fibrils enable an attachment and alignment of at least one type of cells; and said scaffold comprises nucleic acid based molecules that modulate gene expression of the cells attached to the fibrils.

14. A composition of claim 13, wherein the thread-like scaffold has porosity at least 80% with interconnected pores to allow capillary flow along the scaffold; said scaffold has the diameter at least 50 microns and mechanical strength in the thread direction at least 20 MPa.

15. A composition of claim 13, wherein the scaffold is biocompatible biodegradable implant with tunable degradation depending on the level of crosslinking ranging from four weeks to one year.

16. A composition of claim 13, wherein the scaffold is the thread-like aligned collagen scaffold crosslinked by EDC/sNHS.

17. A composition forming a rod-like fibrillar micro-carrier with the fibrils aligned in the direction of the rod such that the fibrils enable an attachment and alignment of at least one type of cells; and said micro-carrier comprises nucleic acid based molecules that modulate gene expression of the cells attached to the fibrils.

18. A composition of claim 17, wherein the rod-like micro-carrier has porosity at least 80% with interconnected pores to allow capillary flow along the micro-carrier; said micro-carrier has the diameter at least 10 microns and mechanical strength in the rod direction at least 20 MPa.

19. A composition of claim 17, wherein the micro-carrier is biocompatible biodegradable implant with tunable degradation depending on the level of crosslinking ranging from four weeks to one year; and a water based suspension of micro-carriers forms an injectable biocompatible biodegradable scaffold.

20. A composition of claim 19, wherein the micro-carrier is the rod-like aligned collagen scaffold crosslinked by EDC/sNHS.
